(19) United States
(12) Patent Application Publication
(10) Pub. No.: US 2016/0352727 A1
(43) Pub. Date: Dec. 1, 2016

(54) SYSTEM AND METHOD FOR ASSET AUTHENTICATION AND MANAGEMENT

(71) Applicant: Reticle Ventures Canada Incorporated, Richmond (CA)

(72) Inventors: Stephen John Day, Richmond (CA); Patrick Quesnel, Ottawa (CA); Geoffrey Clarke, Mississauga (CA); Adrian Chan, Ottawa (CA)

(73) Assignee: Reticle Ventures Canada Incorporated, Richmond (CA)

(21) Appl. No.: 15/161,355

(22) Filed: May 23, 2016

Related U.S. Application Data
(60) Provisional application No. 62/166,317, filed on May 26, 2015.

(51) Int. Cl. H04L 29/06; A61B 5/145; A61B 5/01; A61B 5/024; A61B 5/0402; A61B 5/00

(52) U.S. Cl. CPC H04L 63/0861; A61B 5/0402; A61B 5/6802; A61B 5/01; A61B 5/024; A61B 5/14542; A61B 5/0006

(57) ABSTRACT

An asset management system and biometric security method is disclosed. The asset management system monitors the operational status of living and non-living assets in the field using sensors fitted to, or otherwise associated with, the asset. The operational status of the asset includes proof of life and indications as to the death or destruction. Electrocardiogram (ECG) signals from living assets may be enrolled in a biometric system at one or more heart rates to permit the identification or authentication of the living asset. ECG signal assessments may be used to increase system performance.

### STATEMENT OF RELATED APPLICATIONS

This patent application claims the benefit of and priority from U.S. Provisional Patent Application No. 62/166,317 having a filing date of 26 May 2016.

### TECHNICAL FIELD

The present invention relates generally to the field of asset management in high security environments or for high security purposes. In one aspect, the invention relates to a system and a method for tracking and managing physical assets (which may be living or non-living) and/or for identifying or authenticating assets using physiological and/or non-physiological data acquired from the asset.

### BACKGROUND OF THE INVENTION

Asset management is a critical component of security operations. Whether the asset is a high-value individual, facility, or other target of interest, it is important for security experts to control access to assets and/or be alerted when assets have been moved, compromised, killed, or destroyed.

The term ‘biometrics’ refers to the automatic recognition of an individual based on their physiological and/or behavioural characteristics [1]. Various biometric modalities exist, such as facial recognition [2], iris, retina, fingerprint, vein patterns, gait, speech, etc.

Since biometrics are inherently linked to the user, they are often more difficult to “steal” or counterfeit than traditional recognition strategies (e.g., PIN, passwords, swipe cards, etc.). Biometrics are often thought of as ‘unique’, however, operationally they are not, with law enforcement examiners at times making high profile misidentifications with latent fingerprints [3].

This highlights a fundamental difference between biometrics and traditional recognition strategies. Traditional strategies require a perfect match in order to authenticate a user (e.g., knowledge of the exact PIN, possession of the correct swipe card). On the other hand, biometrics make that decision based on a close match, where the strength of the match is represented by a match score.

An electrocardiogram (ECG) is a measurement of the electrical signals generated by the heart. While the properties of an ECG provide important medical diagnostic information, an ECG can also be used as a biometric security modality.

A healthy individual's heart tends to contract in the same way, which results in a recognizable, repeating pattern within the ECG, called a PQRST complex. The recorded pattern within an ECG is dependent on where the electrodes are placed on an individual's body. The medical community has established standardized electrode locations that are used to obtain various ‘views’ of the heart, called ‘leads’.

While a healthy individual's ECG has the roughly the same PQRST shape for a given lead, inter-subject variability has been documented in the literature [4]-[9]. The exact shape of an individual's PQRST complex is dependent on the electrophysiological properties of their cardiac muscle (i.e., the signal source), such as heart mass orientation and the conductivity of various areas of the heart [7], [9]. Additionally, geometrical and electrophysiological attributes of an individual's anatomy (i.e., the transmission medium between the cardiac muscle and electrodes on the skin, where the ECG is recorded), such as torso shape, position and orientation of the heart within the torso, percentage body fat, position of surrounding organs etc., further affect the recorded signal [6], [7], [9]-[11]. This results in significant inter-individual variation amongst a population.

An ECG is an easily measured physiological trait with potential to be an enhanced biometric security modality. While some general ECG features are common to the entire population, a significant portion of the signal is unique to each individual.

The ECG signal's uniqueness is attributed to the range of physiological factors that influence its properties, including the shape, location, orientation and electrical properties of the heart and the surrounding tissues.

Progress has been made in biometric identification using clean ECG signals in laboratory settings. The following table summarizes some recent studies developing and evaluating ECG biometric modalities over large datasets under ideal laboratory conditions:

TABLE 1
Performance of various ECG biometric modalities:

| Study | Correct Identification (%) | Sample Size |
|---|---|---|
| Zhang (2006) [12] | 85-97 | 502 |
| Wubbeler (2007) [13] | 98 | 74 |
| Silva (2007) [14] | 99 | 168 |
| Chan (2008) [15] | 89-95 | 50 |
| Odinaka (2010) [16] | 77-97 | 269 |
| Shen (2011) [17] | 95 | 168 |
| Wahabi (2014) [18] | 96 | 1020 |

In addition to ECG, a number of other biometric security modalities using criteria accepted in the biometric security industry have been used [19]. These criteria include uniqueness, permanence, performance, circumvention, liveness detection, continuous authentication, collectability and universality. Uniqueness and permanence are viewed as prerequisites for a modality to even be considered for biometric security. The relative importance of the remaining criteria is application dependent.
a) Uniqueness—While the basic pattern of an ECG signal is present in all individuals, there are various individual physiological parameters that alter the waveform to make it unique. The uniqueness of ECG is explained by its inherent inter-individual variability. Uniqueness is a basic prerequisite for a biometric security modality (i.e., without this trait, a modality cannot be used for identification).
b) Permanence—A physiological signal must exhibit little variation over time in order to facilitate biometric matching against previously recorded templates. The permanence of ECG is explained by its intra-individual variability. This is an issue with all biometric security modalities. As a person ages and aspects of their physiology change, a biometric signal may change from the template signal they provided on enrolment. Relative permanence is a basic prerequisite for a biometric security modality (i.e., without this trait, a modality cannot be used for identification).
c) Performance—The performance of a biometric modality primarily refers to its accuracy. Evaluation of performance varies widely across different studies, and is highly dependent on the characteristics of the dataset and the collection and testing methodologies. Despite the difficulties in quantifying performance, this criterion is extremely important to the confidence of an identification method.
d) Circumvention—This trait refers to the ease of “tricking” a biometric system into falsely authenticating an intruder. Even modalities with high performance can be vulnerable to misidentification due to circumvention. While various techniques have been demonstrated to circumvent other biometric security modalities, falsifying an ECG waveform is considerably more difficult. Circumvention of an ECG-based security system would require covertly recording clean ECG using the same electrodes and electrode sites as the security system. This signal would then need to be played back to the system by a purpose-built electronic device, and would require knowledge of the system's signal collection methodology.
e) Liveness Detection—This trait is an extra layer of circumvention prevention that determines whether a biometric signal is coming from a living person. In their most basic forms, modalities such as fingerprints, face recognition and iris recognition may be circumvented using photographs. Liveness detection would prevent this type of attack. An ECG signal has inherent liveness detection, as it is only present in living people.
f) Continuous Authentication—This trait refers to the ability of the modality to re-authenticate the user on a regular basis, rather than merely at point-of-entry. It is mainly dependent on the portability of the sensor, and is related to robustness to circumvention.
g) Collectability—This trait refers to ease of collecting and processing the biometric signal. The ECG signal requires a user to interact with two electrodes for several seconds to record a sample signal. Evaluating collectability depends on the application—sometimes identification must be made based on existing information, without recording new data. ECG data can be recorded using low cost electronic equipment.
h) Universality—An effective biometric modality must be something that can be universally measured from the general population. This property is satisfied by ECG, as it is a vital sign that exists in everyone with a beating heart.

Each biometric modality has its own strengths and weaknesses, which must be considered depending on the deployment application [2]. ECG has the potential to meet all of the above criteria. Nevertheless, two challenges for an ECG-based biosecurity system are “Permanence” and “Performance”.

To be able to differentiate between individuals, an identifier must exhibit high variation between individuals (inter-individual variation) and low variation within repeated measurements for the same individual (intra-individual variation). Thus, inter- and intra-individual variation are prerequisites for a signal to be considered for use as a biometric modality.

The anatomical features that cause inter-individual ECG variability also cause intra-individual stability. Typically, anatomical changes are measured in months/years, thus lending ECG its relative stability over time.

However, physical changes to an individual's anatomy (e.g., heart attack) can sometimes result in (potentially permanent) changes to their ECG. Additionally, changes in heart rate affect the proximity of the T-wave to the QRS complex [20].

In some cases, drug administration can also have the potential to affect ECG morphology [21], however, this is primarily related to sodium channel blockers used to treat cardiac arrhythmias and some anaesthetics. Caffeine, which has been shown to behave as a sodium channel blocker, has minimal effect on ECG morphology in safe doses [22]. Additional intra-individual variation can result from an individual's mental state (e.g., heart rate variability, dilation of blood vessels, etc.) [23], [24], [25].

Although the accuracy of ECG biometrics has been estimated to be about 3% less than that of other modalities (e.g. fingerprints), ECG has certain application specific advantages in that:
a) a person's ECG only exists in living people and is difficult to spoof, making it robust to common attacks on biometric security systems;
b) the authentication of a person's identity can be continuously monitored, unlike other modalities; and
c) cost effective, wearable ECG measurement systems have been developed for the health & fitness market, and they can also be used for security applications

Nevertheless, intra-individual variability poses significant challenges to the effective use of ECG as a biometric modality or means of identification. In particular, low signal quality and variations in ECG patterns over time and at varying heart rates pose challenges for the identification or authentication of individuals using ECG signals.

### BRIEF SUMMARY OF THE INVENTION

The present invention is directed to a system and method for ECG-based biometrics as well as an asset management system for identifying or authenticating assets to control access, verify proof of life, or verify non-destruction of the asset.

In one broad aspect, the present invention is directed to a system for managing one or more physical assets. The system comprises one or more sensors to transmit at least one physical or physiological characteristic of the asset; a computing device (or communication device) for determining at least one condition of the asset based on the at least one physical or physiological characteristic; and an interface for relaying the at least one condition of the asset to an asset manager, operator, or watch keeper. In some embodiments, the system further includes a server in communication with the computing device to relay information to the interface used by the manager/operator. The server, when present, may also have the ability to issue commands to the asset, including enabling or disabling the one or more sensors, changing the settings of the one or more sensors, or issuing orders to the asset.

The assets managed by the system can be living (e.g. people or animals) or non-living (e.g. vehicle, weapon systems, artwork, or objects of value). In some applications, the physical or physiological characteristic of the asset may be an image, video, physical location (e.g. GPS coordinate), temperature, indicia of assembly status (e.g. relative position of one or more sub-components), heart rate, oxygen saturation, or electrocardiogram (ECG) signal. In some cases, the condition or status of the asset is the identity of the asset, the authentication status of the asset, the location of the asset, or whether the asset has been killed or destroyed.

In another broad aspect, the present invention is directed to a method for enrolling, identifying, or authenticating a living asset using an ECG signal as a biometric modality.

In one aspect, the invention pertains to a method of enrolling an individual in an ECG-based biometric security system. In one embodiment, the method comprises the steps of: verifying the identity of the individual using at least one prior authentication factor; acquiring at least one ECG signal from the individual; generating an enrolment template for the individual from the ECG signal; and storing the enrolment template in an enrolment database. In some embodiments, the method includes acquiring the ECG signal at a given heart rate and enrolling the individual in the enrolment database at that heart rate. In such embodiments, an individual may be enrolled at multiple heart rates.

In some cases, the prior authentication factor used to verify the user prior to enrolment is a personal identification number (PIN), password, physical location, Global Positioning System (GPS) coordinate, or a biometric modality. Where the biometric modality is an ECG signal, the individual may be automatically re-enrolled after each successful authentication, at pre-determined time intervals, or in response to the fulfillment of specific conditions dictated by the asset management system.

The invention also provides a method of identifying an individual using ECG-based biometrics. In one embodiment, the method comprises the steps of acquiring at least one electrocardiogram (ECG) signal from the individual at a given heart rate, generating a query template for the individual at the given heart rate from the ECG signal, and comparing the query template to an enrolment database to identify the individual. In this mode, the invention may permit the identification of a previously-enrolled individual on the basis of their ECG signal alone (within a certain measure of confidence) thereby establishing proof of life for that individual.

In some applications, this identification method can also be used in a ‘single-factor’ authentication by performing an identification as described above, and then proceeding to make an authentication decision on the basis of the closeness of the match, to grant access to an asset.

Operationally, ECG biometrics can be fused with other non-biometric or biometric security modalities to increase performance through redundancy and provide robustness to circumvention (e.g., PIN with built-in distress code must be entered on an associated smartphone upon commencement of ECG continuous authentication).

The invention may thereby provide a method of authenticating an individual using ECG-based biometrics. In one embodiment, the method comprises the steps of: verifying the identity of the individual using at least one additional authentication factor; acquiring at least one electrocardiogram (ECG) signal from the individual at a given heart rate; generating a query template for the individual at the given heart rate from the ECG signal; and comparing the query template to an enrolment database to authenticate the individual. In some embodiments, the prior authentication factor is a personal identification number (PIN), password, physical location, Global Positioning System coordinate, or biometric modality, including an ECG signal. In this mode, the invention may permit the authentication of a previously enrolled individual in a two-factor authentication system.

In some embodiments, the invention further includes a signal quality assessment to evaluate the quality of the ECG template generated from the individual before using the template in further enrolment, identification, or authentication actions. If signal quality falls below a defined threshold, the ECG is re-acquired before its use in further steps. Alternatively, the ECG template is generated, but a low confidence score will be assigned to the decisions based on the ECG template. In some applications, particularly where the ECG is gathered using wearable technologies, signal quality assessment may improve the accuracy of enrolment, authentication, and identification in the ECG biometric method.

The invention may also include a heart-rate calculation in the enrolment, identification, or authentication of users to the ECG biometric system. The enrolment of individuals at multiple heart rates permits later comparisons to be made on a heart-rate specific basis, which may increase the accuracy of identifications or authentications in some applications.

In another broad aspect, the invention pertains to an ECG biometrics system for enrolling, identifying, or authenticating a living asset. In one embodiment, the system comprises a sensor for obtaining an ECG signal from the living asset, an enrolment database comprising a plurality of ECG templates, and a computing device (such as a mobile communications device) programmed to perform the methods described above. In some embodiments, the enrolment database is on a server accessed by the computing device over a communications network, such as a Local Area Network (LAN) or the Internet.

In another broad aspect, the invention pertains to a method of identifying a living or non-living asset. The method comprises acquiring at least one signal, via one or more sensors, from the asset; transmitting the signal from the one or more sensors, via a transmission link, to the processing device; generating a query template for the asset, by a processing device, from the signal; comparing the query template, by the processing device, to templates with templates stored in an enrolment database; generating a similarity score, by the processing device, for each of the templates stored in the enrolment database; and if a highest similarity score generated is within a predetermined range, identifying, by the processing device, the asset as an enrolled asset associated with a stored template having the highest similarity score. The signal may be an ECG signal at a given heart rate.

In another broad aspect, the invention pertains to a system for managing one or more living or non-living assets, the system comprises one or more sensors for sensing at least one physical or physiological characteristic of the one or more assets; and a computing device for receiving the sensed at least one physical or physiological characteristic from the one or more sensors, and for determining at least one condition of the asset based on the at least one physical or physiological characteristic, wherein if the at least one condition of the asset is determined to meet a threshold, the computing device issues an instruction to the one or more sensors or to the asset or issues an alert message.

In another broad aspect, the invention pertains to a non-transitory computer readable medium containing instructions to perform the steps of acquiring at least one signal, via one or more sensors, from the asset; transmitting the signal from the one or more sensors, via a transmission link, to the processing device; generating a query template for the asset, by a processing device, from the signal; comparing the query template, by the processing device, to templates with templates stored in an enrolment database; generating a similarity score, by the processing device, for each of the templates stored in the enrolment database; and if a highest similarity score generated is within a predetermined range, identifying, by the processing device, the asset as an enrolled asset associated with a stored template having the highest similarity score. The signal may be an ECG signal at a given heart rate.

The continuum of applications for ECG biometrics and asset management is vast—ranging from the financial community to medical devices to counter-terrorism efforts.

In an aspect the invention a provides a method of enrolling a living or non-living asset in an asset security system, the method comprising the steps of: with a processing device, verifying the identity of the asset using at least one prior authentication factor; acquiring a signal, via one or more sensors, from the asset; transmitting the signal from the one or more sensors, via a transmission link, to the processing device; with the processing device, associating the signal with the identity of the asset; using the processing device to generate an enrolment template for the asset from the signal; and storing the enrolment template in an enrolment database.

In another broad aspect the invention provides a method of authenticating a living or non-living asset, the method comprising the steps of: with a processing device, verifying the identity of the asset by comparing a first authentication factor with a prior authentication factor; if a match of the first authentication factor and the prior authentication factor is found, acquiring a signal from the asset, via one or more sensors; transmitting the signal from the one or more sensors, via a transmission link, to the processing device; with the processing device, generating a query template for the asset from the signal; with the processing device, comparing the query template to templates stored in an enrolment database and associated with the asset, verified by the at least one prior authentication factor; using the processing device to generate a similarity score for each of the templates stored in the enrolment database; and if the highest similarity score generated is within a predetermined range, authenticating, with the processing device, the asset as an enrolled asset.

The invention may also provide a biometric security system comprising one or more sensors; a processing device in communication with the one or more sensors and an enrolment database, the processing device comprising a processor, wherein the processor is configured to: verify the identity of an asset by comparing a first authentication factor with a prior authentication factor; if a match of the first authentication factor and the prior authentication factor is found, receive and acquire a signal from the asset, the signal sent via a transmission link from the one or more sensors; generate a query template for the asset based on the signal received; compare the query template to templates stored in the enrolment database and associated with the asset verified by the first authentication factor; generate a similarity score for each of the templates stored in the enrolment database; and if the highest similarity score generated is within a predetermined range, authenticate the asset as an enrolled asset.

### BRIEF DESCRIPTION OF THE DRAWINGS

FIG. 1 is a stylized ECG reading showing the various segments of one complete heart beat (PQRST complex).

FIG. 2 is a set of two PQRST complexes taken from two healthy subjects.

FIG. 3 is a comparison of various biometric modalities.

FIG. 4A is a series of PQRST complexes recorded from eight subjects, several years apart.

FIG. 4B is a set of PQRST complexes recorded from a single subject, at 144 beats per minute (left) and 186 beats per minute (right).

FIG. 4C is an illustration of the effect of various heart rates on a single individual's ECG measurements.

FIG. 5A is an overview of an asset management system according to an embodiment of the present invention.

FIG. 5B is an overview of different data abstraction levels in an identification decision making process, based on the data collected from the sensors.

FIG. 5C is a specific embodiment of one asset management system according to the present invention.

FIG. 6 is an overview of an ECG biometric method according to the present invention.

FIG. 7A is an ECG template generation method according to the present invention.

FIG. 7B is a set of ensemble averaged PQRST complexes collected from clean ECG data.

FIG. 7C is a set of ensemble averaged PQRST complexes collected from noisy ECG data.

FIG. 7D is a specific embodiment of an ECG template generation method according to the present invention, in which templates are generated in a heart-rate specific manner.

FIG. 7E is an ECG template generation method according to the present invention in which a signal quality assessment is enforced.

FIG. 7F is an ECG template comparison method according to the present invention in which the comparison is made in a heart-rate specific manner.

FIG. 8A is a method of enrolling an individual in an ECG biometric security system according to the present invention.

FIG. 8B is a specific embodiment of a method of enrolling an individual in an ECG biometric security system, which implements a signal quality enforcement and heart-rate specific comparison.

FIG. 8C is an illustration of the enrolment of an individual's ECG template.

FIG. 8D is a scatter plot illustrating the effect of heart rate on the ability of the system to match a test template against an enrolled template.

FIG. 9A is a method of identifying an individual using an ECG biometric security system according to an embodiment of the present invention.

FIG. 9B is a specific embodiment of a method of identifying an individual using an ECG biometric security system, which implements a signal quality enforcement and heart-rate specific comparison.

FIG. 10A is a method of authenticating an individual using an ECG biometric security system according to an embodiment of the present invention.

FIG. 10B is a specific embodiment of a method of authenticating an individual using an ECG biometric security system, which implements a signal quality enforcement and heart-rate specific comparison.

### DETAILED DESCRIPTION OF PREFERRED EMBODIMENTS

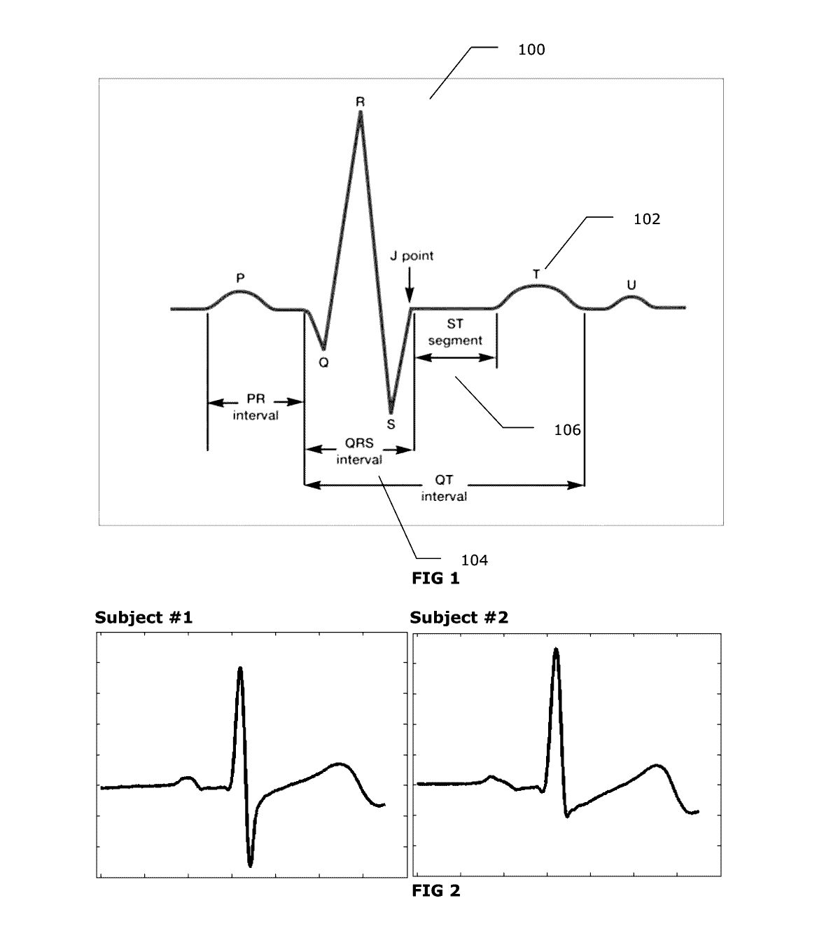

An ECG reading includes various segments of one complete heart beat, as shown in the PQRST complex 100 in FIG. 1. Different individuals have different PQRST complexes. FIG. 2 shows an example of inter-individual variation of PQRST complexes.

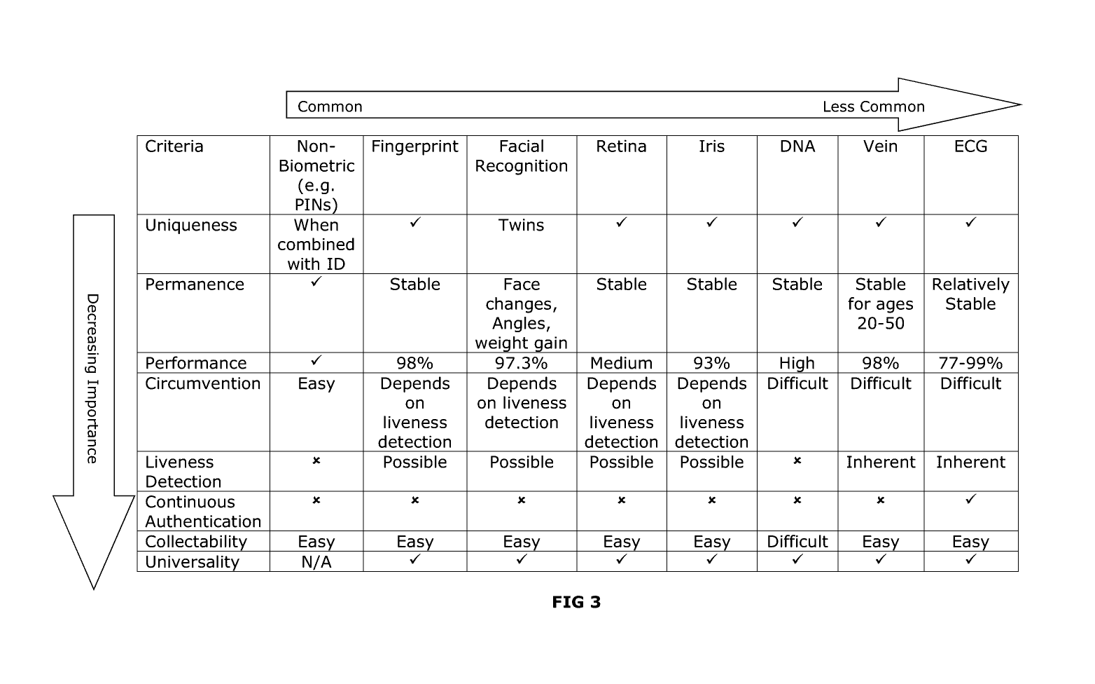

FIG. 3 represents a comparison of ECG to other biometric security modalities using criteria accepted in the biometric security industry. FIG. 3 shows that ECG has the potential to meet all of these criteria.

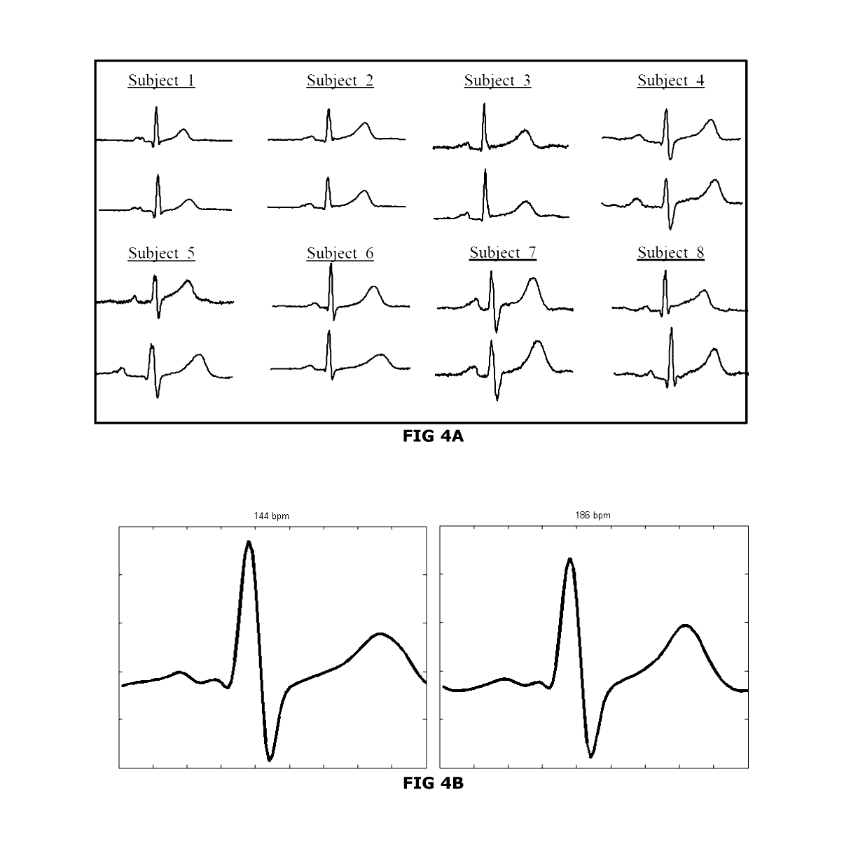

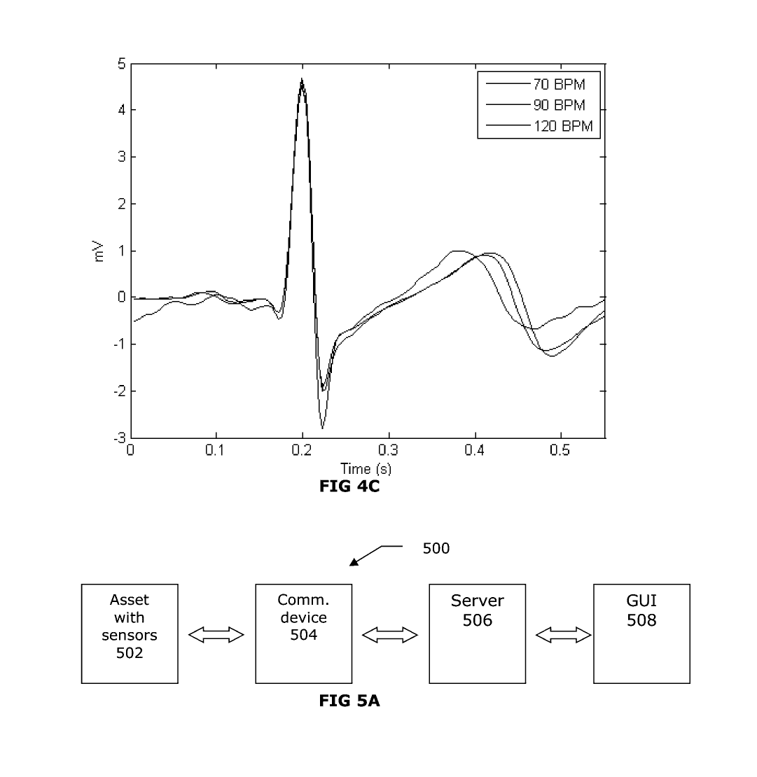

The PQRST complexes of an individual's ECG are generally stable over years as shown in FIG. 4A. However, physical changes of the individual anatomy may result in changes to its ECG, as shown in the ECG of subjects 5, 6, and 8 in FIG. 4A. Changes in heart rate affect the proximity of the T-wave 102 to the QRS complex 104, as illustrated in FIGS. 4B and 4C.

### Asset Management System:

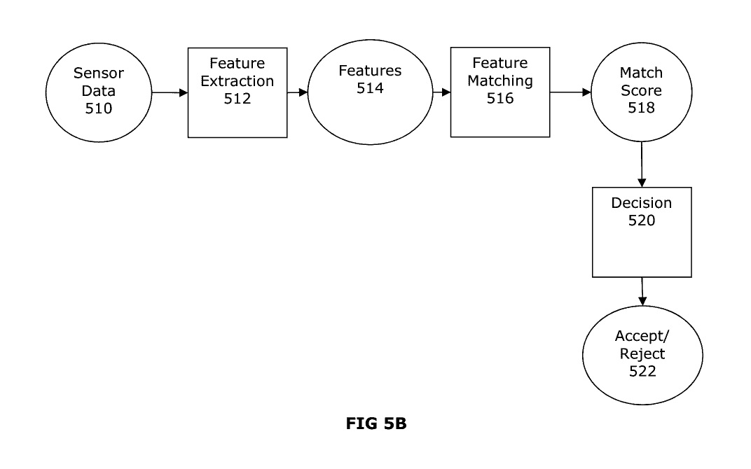

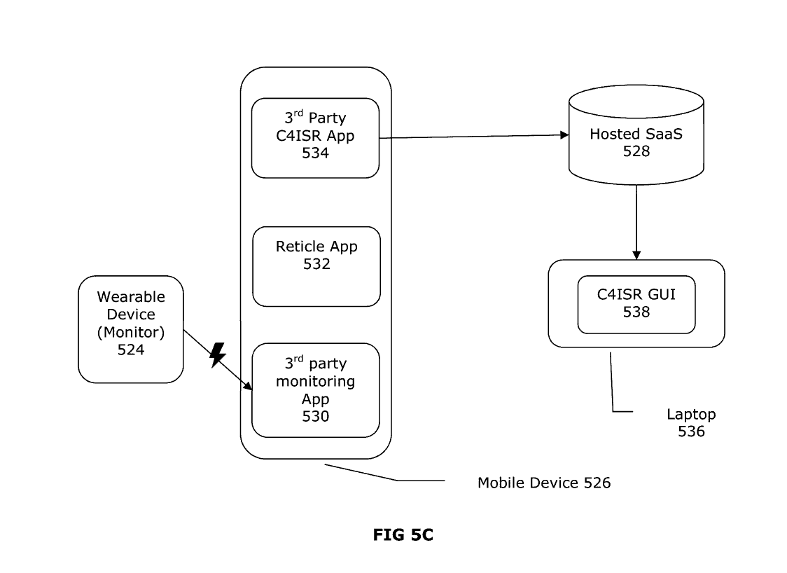

FIG. 5A provides an overview of an asset management system 500 that provides medical, authentication, location, and other information for real-time or time-delayed tracking of living and non-living resources (i.e. “assets”). Briefly, the system comprises an asset equipped with sensors 502, a communication device 504 to aggregate and transmit sensor data, a server 506 to aggregate and store data, and a command and control graphical user interface 508 (GUI). In other embodiments, the communication device 504 and server 506 may be integrated into a single unit, which may also include the GUI. Each of the communication device 504 and the server 506 may include a processor and a memory.

FIG. 5B provides a biometric system for processing data at a given abstraction level and presenting it at a higher level of abstraction according to an example embodiment. For example, the raw ECG data represents data at the lowest level of abstraction. An algorithm processes this raw data and extracts a heart rate. The heart rate data is at a higher level of abstraction—it contains more pertinent information than the raw ECG signal but less data overall. For a biometric system, the sensor data 510 which may be raw ECG data is the first level of abstraction. The sensor data 510 may be extracted at step 512 to obtain extracted features 514 which may be for example the distance between QRS and T waves and which are at a higher abstraction level. The features are then matched at step 516 and a match score 518 is generated, which represents the next highest level of abstraction. The decision step 520 represents the highest level of abstraction; it contains less information than any of the other steps, but its information is much more pertinent. Based on the decision step 520, the biometric system may then accept or reject the user at step 522.

FIG. 5C provides a specific example of an asset management system according to one embodiment of the present invention. Here, the asset is a person equipped with a wearable device 524, such as a commercially available base-layer compression shirt for fitness applications. The wearable device 524 comprises sensors for collecting data related to the individual wearing the wearable device 524. The wearable device 524 is equipped with communications technologies, such as a direct transmitter, or Bluetooth™ technology for communicating with a communications device 504, such as a mobile device 526. The data collected by the sensors include physiological data, such as real-time heart rate (HR) and breathing rate (BR) vitals on the individual wearing the wearable device 524, including an ECG signal. The communication device 504 then transmits the collected data to a hosted platform server 506, for example, a hosted SaaS 528, via a communication link of a communication networks, such as, the Internet, wireless communications networks, or wired communications networks. In this embodiment, the communication device 504 runs particular software designed for this purpose. The data collected by the sensors of the wearable device 524 may be further combined, either by the communications device 504 or the server 506, with other data collected from third party applications, such as video, audio, text chat, and other data streams, including 3^{rd} party monitoring apps 530, Reticle App 532 or a 3^{rd} Party C4ISR App 534 depending on the available application program interfaces (“APIs”) and other ingest parameters of the third party application. The server 506 can then be accessed by an operator (or “watch keeper”) in this case remotely or locally, for example, using a laptop 536, via a graphical user interface that permits the operator to monitor the various data streams and send commands to the asset. The nature of the commands will be dependent on the type of asset being controlled. Commands will vary according to the type of asset. If the asset is a living asset, for example a person, the commands may be sent through the communication device, for example, a cell phone, and can include various types of instructions, such as, leave-the-area, proceed, etc. If the asset is a non-living asset, such as a building, weapon system, etc., commands can instruct the asset, for example, through wireless communications, to enter an armed-state, to allow access, to disallow access, to activate certain sensors, etc.

### Assets

A wide variety of assets may be managed with the system, including, but not limited to: personnel, animals (e.g. high value race horses, cattle, or pets), vehicles (both civilian and military), weapon systems (e.g. nuclear warheads), buildings, facilities, and personal property (e.g. art). In some embodiments, the asset is a military vehicle (e.g. ship, tank, transport, aircraft, etc) or weapons system (e.g. missile system, nuclear weapon, etc), the access to which is restricted to certain individuals and the location of which is restricted to certain areas.

Where the asset is an individual or animal, the system may be used to identify or authenticate the asset, as well as to establish proof of life. For example, an authenticated ECG signal may be used to confirm that the asset is alive.

Where the asset is a vehicle, building, or other inanimate object, the system may be used to secure access to the asset, as well as to establish that the asset has not been tampered with or destroyed. For example, a series of proximity sensors may be positioned around the inanimate object, with an increase or decrease in proximity indicating a state of disassembly or destruction of the asset. In other applications, commercially available tipping or tamper sensors may be used to determine whether the inanimate asset is intact. For example, the inanimate asset is intact if two GPS co-ordinates associated with the inanimate is a fixed distance apart. If the inanimate asset is intact, proof of life of the inanimate asset is also established. As well, ECG biometrics may also be used to grant or deny access to the asset.

### Sensors

A wide variety of sensors may be associated with a particular asset to monitor its status including, but not limited to: GPS systems, inertial measurement units (accelerometer/gyro/magnetometer), force sensors, proximity sensors, thermometers, barometers, altimeters, weather stations, gas analyzers, microphones, cameras, and various physiological sensors (e.g. ECG, respiration rate, oxygen saturation, body temperature). These sensors collect raw data from an asset of interest. The collected raw data is transmitted to a communication device regarding the status of the asset. The raw data may be initially processed, for example, by data processing and machine learning algorithms to present data to the communication device 504 at a higher level of abstraction.

The status of the asset may include various types of information, including location, altitude, speed, direction, and temperature. For living assets, status information may include an indication as to proof of life, such as an authenticated ECG signal. For non-living assets, status information may include information indicative of tampering, disassembly, or destruction. For example, a change in relative position in a plurality of proximity sensors distributed across the asset may indicate that the asset has been disassembled or destroyed.

In embodiments where the asset is a person, various wearable devices may be used. A number of suitable wearable devices are now commercially available. This includes base-layer compression shirts which provide data on respiration and heart rate, including ECG signals. Chest straps, portable medical monitors, pressure garments, wristbands, bracelets, watches, and the like are also contemplated, as are all other forms of wearable sensors. Sensors may also be carried on personal effects ordinarily carried by the person, such as a portable communication device or a mobile phone.

### Communications Device

In the embodiment shown in FIG. 5A, the communication device 504 aggregates raw data collected by sensors and sends it to a central server 506. The communication device 504 may transmit the raw data to the server 506, or use data processing and machine learning algorithms to present data at a higher level of abstraction. For example, the raw ECG represents data at the lowest level of abstraction. The raw data may be processed and a heart rate may be extracted from the processed data. The heart rate data is at a higher level of abstraction as it contains more pertinent information than the raw ECG signal, but less data overall. In the case of a biometrics system, abstraction levels can be generally classified as follows:
Actual sensor data (e.g., raw ECG)
Extracted features (e.g., slope of various parts of the wave, distance between QRS and T waves, etc.)
Score (e.g., resulting score from comparing the query template to the database)
Decision.
   In this case, the decision step represents the highest level of abstraction; it contains less information than any of the other steps, but its information is much more pertinent. Algorithms may be used to process the data at a given abstraction level in order to produce the next higher abstraction level.

In some embodiments, the server 506 may also issue commands to the asset through the communication device 502. This can include enabling or disabling sensors, changing the settings of sensors, or issuing instructions to the asset. In some aspects, the communication device and the sensors may be integrated into a single device. In other aspects, the sensors on the asset are configured to communicate with a communication device close to the asset, for example, via near field communications such as Bluetooth™. Where the sensors on the asset are configured to communicate with the communication device close to the asset, a control module is built in the sensors. In the case of wearable ECG, a control module samples the signals collected by electrodes, stores the signal on the control module locally, and transmits it via Bluetooth™ to a communication device, such as a cell phone.

Typical commands sent to the sensor control module by the server 506 vary with the type of asset. They may include adjusting the sampling frequency of the sensors, or requesting the communication device 501 to send various types of information. They may also include commands/directions to a person via the communication device 504 (e.g., cell phone) or adjust the sampling rate of the ECG sensors via the sensors' control module. They may also include instructions to a door to grant access, or to a GPS to change its update frequency, etc.

A wide variety of communication devices could be used for this purpose, including, but not limited to: personal computers, single board computers, tablet computers, cell phones, smart phones, WIFI enabled devices, Bluetooth™ enabled devices, satellite enabled devices, and RF enabled devices.

In FIG. 5C, the mobile device 526 may be a smart phone running a custom software application. In other embodiments, the role of the communications device 504 and the server 506 may be combined into a single device, so as to eliminate the need for transmission of data over a communication network. The single device combined communication device 502 and server 506 communicates with the wearable device or with the sensors of the wearable device in the same manner as the communication device 502. In an embodiment, the wearable device may include a control module that collects (samples) data from the sensor of the wearable device. The control module may interact with external devices, for example, a computer via USB connection, or a cell phone via Bluetooth™ connection. As well, the control module may be capable of data processing. In another embodiment, the control module includes a memory to store data, such as a built-in storage to record and store data. Data processing may vary depending on the type of data and type of asset. For example, in the case of ECG data processing can include but is not limited to filtering, heart rate calculation, signal quality assessment, template generation, and authentication. The data may be processed on any of the devices in the signal chain, such as the control module of the wearable device, the communication device 504, the server 506.

### Server

In the embodiments shown in FIGS. 5A and 5C, a server 506 or 528 aggregates and stores data streams originating from one or more assets. The server 506 or 528 can be deployed in a local network or over the internet. The server 506 or 528 includes software to accept communication streams from a variety of devices. The data may be stored as received in a database in the server 506 or 528, or further processed and stored at a higher level of abstraction. The data may be further processed in a similar manner as processed by the communication device 504. Data processing steps will vary depending on the type of data and type of asset. In the case of ECG data processing can include but is not limited to filtering, heart rate calculation, signal quality assessment, template generation, and authentication. These processing steps can occur on any of the devices in the signal chain, such as the control module of the wearable device, the communication device 504, the server 506.

The server 506 or 528 may also include software to implement authentication in order to avoid access by unauthorized devices. The server may implement data processing and machine learning algorithms to aid in decisions based on the data. In an embodiment, the machine learning algorithms identify the asset and the location of the asset, and calculate information about the current state of the asset from raw data collected from the asset. In one example, functions of the algorithms include assessing GPS signals from positions that are a known distance apart on the asset to determine if the asset is intact (generally for non-living assets), calculating heart rate from raw ECG, combining heart rate and breathing rate to assess a human asset's current physical state, and authenticating an individual human or living asset with a heart beat based on their raw ECG data.

In some embodiments, the server 506 or 528 can also be used to issue commands to assets through the communication device(s). The commands to an asset depend on the type of the asset, such as a living asset or a non-living asset. Commands may be automatically issued in response to criteria set by a watchkeeper/operator. Alternatively, the commands may also be issued at the discretion of the watchkeeper/operator based on the data collected from the asset. An example of an automatic command includes turning on cameras or changing their viewing angle when another asset enters a restricted area. The commands issued by the server 506 or 528 will pass through the communication device 504 to the target asset. In an embodiment, a control module of the target asset will control the target asset in accordance with the commands received, such as controlling the sensors, cameras, etc.

This can include enabling or disabling sensors, changing the settings of sensors, or issuing instructions to the asset. If the asset is a living asset, instructions can be sent to a communication device, such as a cell phone, which will notify the asset with the new instructions. If the asset is a non-living asset, such as a wearable ECG device, exemplary commands may include instructing the device to report and update the information on the server more frequently.

### GUI

The data collected by the sensors and received by the server 506 or 528 can be displayed on a command and control GUI 508, which is communicably connected with the server 506 or 528. The command and control GUI 508 is the interface through which an operator/watchkeeper can interact with data collected in real time or data stored in the database of the server 506 or 528, preferably via a customizable display of incoming data.

The server 506 or 528 analyzes or processes the data collected by the sensors and displays it using selected display formats. Display formats include, but are not limited to: raw data, tabular display, map overlay, and alerts-based display. Based on the analysis or processed results of the collected sensor data, the server 506 or 528 may generate alerts implementing complicated logic.—For example, an alert can be triggered automatically or manually if an asset is in a specific location at a specific time with a heart-rate above a specific threshold Criteria used to trigger the alerts are dependent on the type of sensor used. If human asset vital signs are monitored, then alerts can be generated if the heart rate goes too low/high, or if the breathing rate stops, etc. If the asset is equipped with GPS, alerts can be generated if asset enters/exits predefined areas. After alerts are generated, the watchkeeper/operator is notified and can take appropriate action, such as assembling rescue forces. Based on the data collected, the alerts are highly configurable to the user's situation and needs.

The GUI 508 may implement options for operators to issue commands to assets, including: enabling or disabling sensors, changing the settings of sensors, or issuing instructions to the asset.

The GUI 508 may implement a user permissions system, where various users have customizable access to specific assets or data streams. For example an operator can only view assets under its direct control, whereas a commander can see all assets within the organization. This can include implementation of more complicated logic, which may be a collection of criteria/rules that must be met. In an example, an operator can only access to the collected data of the asset and control the asset if the asset is in a specific location during a specific time frame. In another example, a commander can allow an operator access to any asset within a defined geographical boundary. Thus, permission settings for any asset entering/leaving the area must be adjusted to include/exclude that operator, as appropriate.

The GUI 508 may display the collected data together with aggregated data from other data sources unrelated to the collected asset data streams. For example, assets may be presented on a map overlaid with traffic and weather data. The other data sources may be pre-stored in the server 506 or 528 or in other servers, and may be accessible by the GUI 508 via the server 506 or 528.

In some embodiments, the GUI 508 is integrated with the server 506 or 528 or communication device 504. In other embodiments, such as the one depicted in FIG. 5C, the GUI may be a C4ISR GUI 538 which is presented on a further device, such as a laptop, 536, which is used by the operator to access the server 506 or 528 locally or remotely via a local area network or a secured connection on the Internet.

### Data Processing and Abstraction

The system presents many opportunities for data processing and abstraction. The system may implement a wide variety of data processing strategies, with the goal of presenting human readable, operationally relevant information to the end users. The data processing and abstraction can be performed at a control module communicably coupled to the sensors 502, the communication device 504, or the server 506.

For example, an operator may wish to be alerted when an asset's heart rate is above a specific threshold. A physiological sensor produces a raw ECG signal. This signal needs to be filtered, processed to calculate the asset's heart rate, and checked against the threshold. In an embodiment, these steps can occur all on one device at the sensors 502, in the communication device 504, or on the server 506. The control module of the sensors 502 may process the data collected by the sensors 502, extract the indicators of interest, and compare the extracted indicator with relevant threshold. Based on the comparison results, an alert may be generated and transmitted to the server 506. The communication device 504 simply passes the alert on to an operator on the side of the server 506. One or more of these steps may also be performed separately on one or more of the control module communicably coupled to the sensors 502, the communication device 504, or the server 506. For example, the initial ECG processing could be done at the sensor level, heart rate detection based on the ECG in the communication device, and checking heart rate against the threshold in the server or GUI.

Furthermore, the system allows for machine learning algorithms to be implemented along the chain. This may enable more intelligent alerting systems, where a general baseline state of an asset is determined. The type of baseline state will vary depending on the asset. Typically, the baseline state of GPS equipped assets will be a geographical boundary which the asset does not usually leave. For human assets outfitted with wearable physiological monitors, the baseline state will be typical heart rate, breathing rate, typical and accelerometer values. Regardless of the type of baseline, all will be calculated by assessing the average value of the metric, and some factor of its standard deviation. Alerts may be generated on any deviation from the baseline by observing either a very large or sudden deviation from what is considered to be an acceptable range. Where the acceptable range is calculated by assessing the average value of the metric in question, and some factor of its standard deviation. Based on the collected position data of an asset by a GPS, if the asset has a pre-determined regular route of travel, and if the collected position data shows that the travel route of the asset is deviated from the pre-determined regular route, the system could trigger an alert.

### Biometric Identification or Authorization:

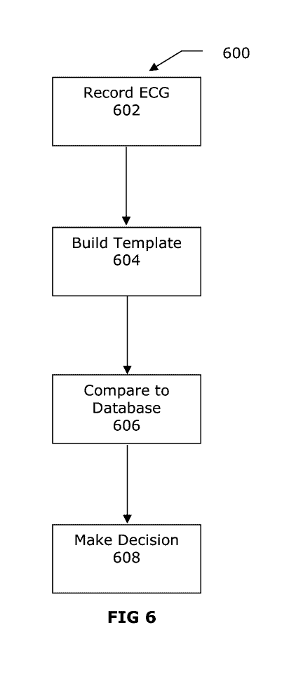

An overview of the basic steps involved in the identification or authorization of an individual using ECG modalities is provided in FIG. 6. Each step will now be discussed in turn.

### Record ECG

The first step of the process 600 involves recording and initial processing of the ECG signal 602. Recording the ECG can be done by measuring a voltage difference between any two points on the skin where a portion of the electrical path between the points crosses the heart.

The voltage signal may be measured using wet or dry electrodes, with or without the aid of electrolyte gel. These electrodes may be used in a variety of configurations, including, but not limited to: stuck directly to the skin, mounted in a shirt or other item of clothing, mounted in a chest strap, or mounted in any device that a person can touch. In some embodiments, the ECG signal is acquired using a sensor embedded in a smart phone or other communication device or a wearable sensor such as a chest strap, undershirt, pressure garment, portable medical monitor, or wristband.

The recorded voltage difference is amplified using an instrumentation amplifier, sampled by an analog to digital converter, and stored as a digital signal. The device that is connected to the electrodes may perform all three of these functions. For example, if an ECG signal is acquired using a sensor embedded in a smart phone, the smart phone will typically contain the instrumentation amplifier, and processor/control module to run the A/D converter and storage. Conversely, all wearable devices may have their own wearable control module/processor that performs the amplification, sampling, and storage (often collectively referred to as the ‘sensor’ or ‘wearable device’). The wearable control module preferably has enough processing power to filter the signals, and run algorithms to extract heart rate (HR), breathing rate (BR), etc. from the raw signals/data collected from the sensors. Typically these HR/BR/etc signals, which are at a higher level of abstraction than the raw physiological signals/data, are sent via near field communications, such as Bluetooth™ to the communication device 504, which in turn sends them out to the server 506 wirelessly, for example, via GSM/wifi.

These signals may be further filtered to remove signal noise. Depending on the processing power of each of the devices, filtering may occur at any stage in the signal flow, for example, at the control module of the sensors 502, at the communication device 504, or at the server 506. In an embodiment, filtering occurs at the sensor-level if the ECG is acquired using a wearable device 524. If the communication device 504 is a cell phone, it may be able to run/perform more computationally intense algorithms/filtering. Alternatively, the raw signals/data may be passed all the way up to the server 506, which can run all of the filtering, and algorithms required. The filter can be a low-pass filter, a band-pass filter, a high-pass, adaptive filters, or a combination thereof.

### Build Template

While the ECG is being continuously recorded, this step attempts to build a generalized template representative of the person's ECG signal. The template generation step 604 may be implemented at any point in the asset management system, including the ECG recording device, an associated communication device 504, or a central server 506 or 528.

In some embodiments, the template building process begins with an assessment of the ECG signal quality. A variety of environmental factors can add noise to the ECG signal. This noise has a negative impact on the ability of the ECG biometric method to make an accurate identification or authentication decision. If the signal is deemed to be of sufficient quality to make an identification or authentication decision the method automatically detects successive individual heart beats, aligns them in the time domain, and averages them to generate a representative template of the ECG waveform. If the signal is not of sufficient quality the template is not generated and no identification or authentication decision is made.

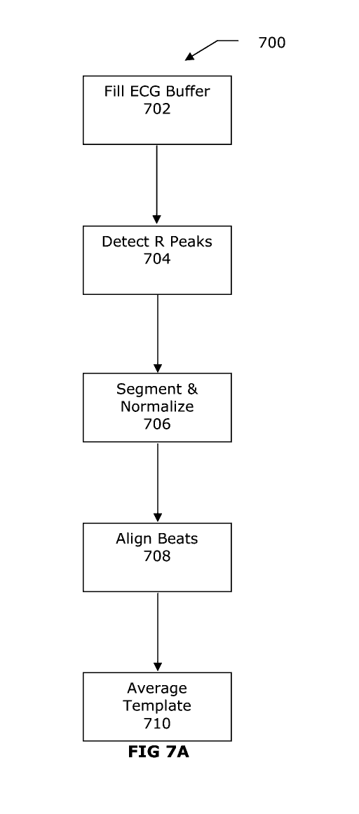

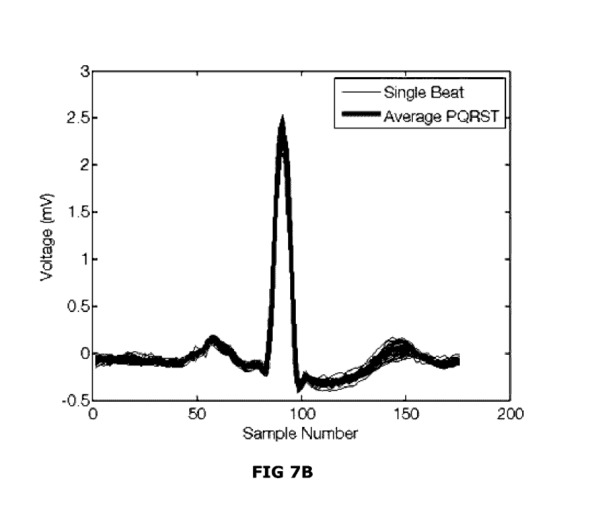

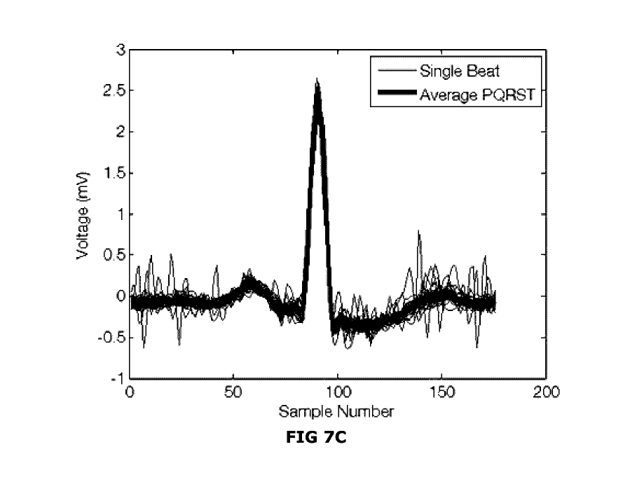

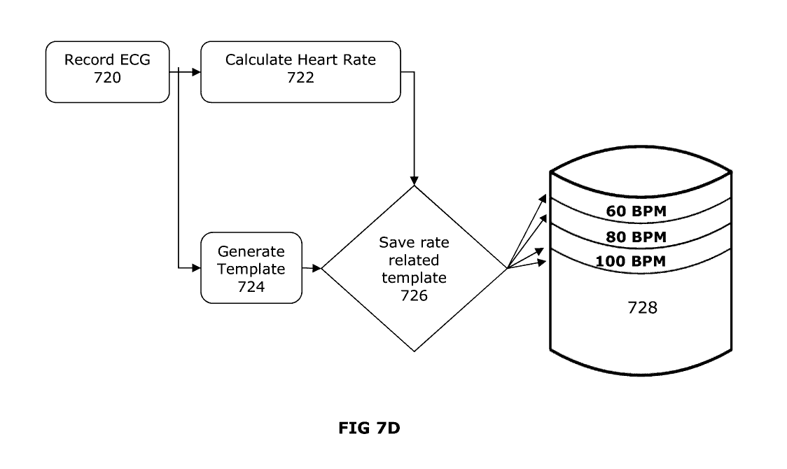

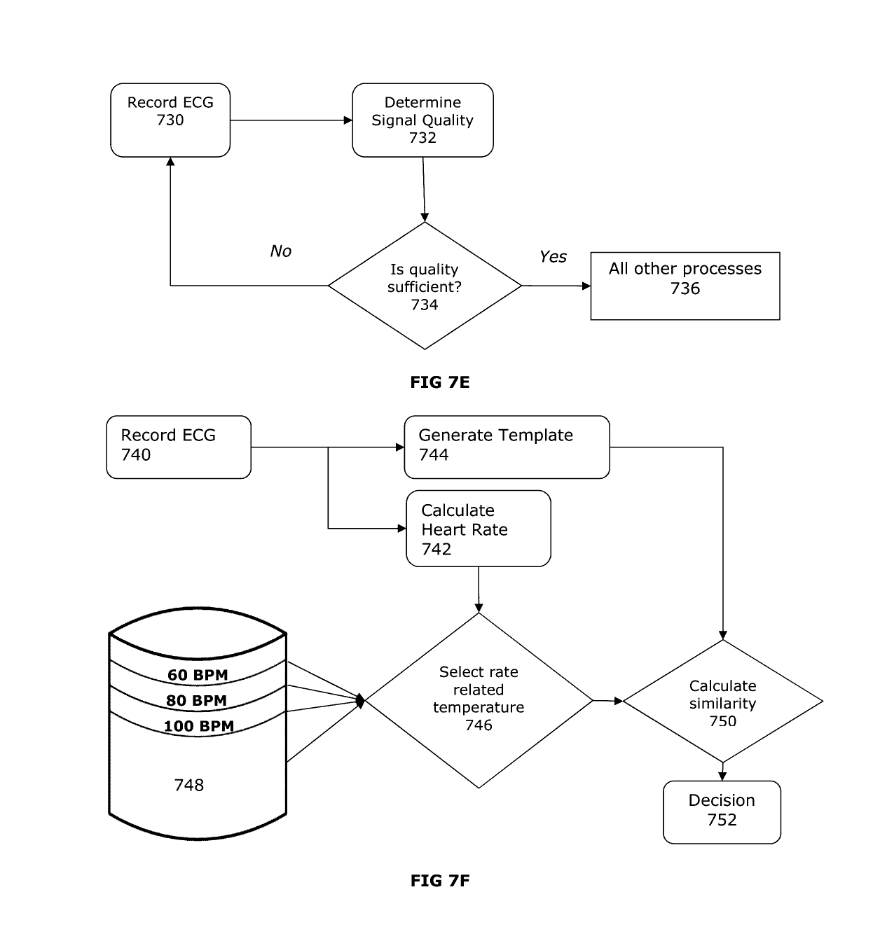

FIG. 7A provides a template generation method 700 according to one embodiment of the invention. In this example, an ECG signal buffer is filled 702 with ECG signal data from the individual. The R peaks of each ECG beat are then detected 704 using QRS complex detection. A segmenting window based on time is then generated around each R peak to extract each complete heart beat. Each beat is then normalized 706 in amplitude and the R peaks are aligned 708 in the time domain. The amplitude of a given heart beat (PQRST complex) will depend on many factors, one of which is the conductivity of the junction between a user's skin and the electrodes. This conductivity may be different each time a user touches the electrodes and may even drift while the user is continuously touching the electrode due to sweat build-up. The amplitude can be normalized to account for this variation through any number of processes. For example, all samples can simply be divided by the maximum sample value.

In an embodiment, the amplitude is normalizing by dividing all sample values that make up the heart beat by their standard deviation. An average ECG beat template is calculated 710. In an example, within a 30 second analysis window, each PQRST complex (i.e., each heart beat) is isolated into a 0.7 second segment, Wₘ[n], which is centered on the R peak. Wₘ is the m^{th} complete beat in the analysis window and n represents the individual sample number (n={1, 2, N}). For a sampling of 250 Hz, this corresponds to segments of N=176 samples; 87 from before the R peak, the R peak itself, and 88 samples after the R peak. The first and last beats in the analysis window are checked to ensure they have the necessary number of samples before and after the R peak. Incomplete PQRST complexes are excluded from the analysis. The action potentials that cause the heart to beat are all-or-nothing phenomena; the electrochemical changes occurring within the tissue will, once triggered, always react in the same way. This means that at each time a subject's heart beats, the resulting PQRST complex should be identical. Small morphological changes to the PQRST complex do occur in healthy ECG because of HR changes, such as shortening of the ST segment. However, given the relatively short length of analysis windows (30 seconds), these changes are assumed to be negligible. Thus, the ensemble averaged PQRST template should represent the ideal PQRST complex, assuming noise to be uncorrelated. Once all PQRST complexes have been segmented and aligned following the previous description, each of the 176 samples is averaged across all beats to get an ensemble averaged PQRST template, W_{avg}[n], as described below.

$${W}_{avg} [ n ] = \frac{1}{M} {\sum }_{m = 1}^{M} {W}_{m} [ n ]$$

where M represents the total number of complete beats in the analysis window.

FIG. 7B illustrates ensemble averaging all complete PQRST complexes in 30 seconds of clean ECG data, while ensemble averaging of noisier ECG data is illustrated in FIG. 7C. The template in this example is a complete heart beat amplitude waveform within a period/segmenting window.

In some embodiments, templates are generated in a heart-rate specific manner, an example of which is provided at FIG. 7D. Here, a template is associated with its specific heart rate. This permits multiple templates to be generated for a single individual, at different heart rates. This also allows ECG signal templates or a heart beat waveform to be compared at the same or similar heart rates as illustrated in FIG. 4C, to reduce intra-individual variation. In this embodiment, the ECG of a target individual is recorded at 720 and the heart rate is then calculated at 722 based on the ECG data. Multiple templates may be generated at 724 and may be saved at 726 in a database 728 of a single individual at various heart rates.

Poor ECG signal quality can impair the ability of the system to enroll, identify, or authenticate individuals. In some embodiments, the system includes a signal quality enforcement step by developing an average template for a segment of an individual, which is run before allowing a template to be used by the system. If signal quality is poor, the ECG signal may be re-acquired from the asset and signal quality re-assessed for the new signal.

FIG. 7E provides an example of a signal quality enforcement according to the present invention. Here, after the ECG is recorded at 730, a signal quality analysis 732 is performed by developing an average template for a segment of an individual, as described previously. This template is then subtracted from each individual beat that contributed to the template 734. If the remaining signal is relatively high powered, the segment is noisy and will be discarded. If the remaining signal is low powered, the segment is clean and a template is generated from the segment and the remaining processes proceed at 736. Generally, a segment is rejected or accepted based on a signal-to-noise ratio (SNR). The segment may be accepted as a template if the SNR is at least 10 dB. However, acceptable SNR value may be varied to reflect a user's requirements.

### Compare to Database

The comparison step 606 analyzes a template and compares it to previously recorded templates stored in a database of enrolled individuals. The template to be compared has the same period as the previously recorded template. This comparison is done using an approach that evaluates closeness of match between two signals. Closeness of match can be evaluated by a variety of methods, including cross-correlation, wavelet distance, or principal component analysis followed by Euclidian distance or Mahalanobis distance.

The comparison of the closeness of match is performed between two comparable ECG waveforms, for example, both ECG waveforms have the same heart rate with the same time period. The database of enrolled individuals may contain multiple template signals per individual, to account for natural variations in the ECG waveform, such as, changes in heart rate that can change the shape of the ECG waveform. If a template is recorded at a high heart rate and compared to an enrolled template from a low heart rate, identification/authentication accuracy may decrease. To compensate, each individual in the enrolled database may have a variety of templates recorded at a variety of heart rates. FIG. 7F provides one such example, in which the heart rate of the query template is calculated and used to retrieve templates at the same or similar heart rate from an enrolment database. In this embodiment, various ECG readings are recorded 740 from which the resultant heart rates may be calculated 742. A template generation step 744 as described before may also be performed. Alternatively the heart rate of the query template may be calculated 742 and used to retrieve templates at the same or similar heart rate 746 from an enrolment database 748. The similarity of the templates may be calculated at step 750 and used to make a decision 752.

For identification applications, closeness of match may be evaluated between the template in question and all of the templates in the enrolled database 748. For authentication applications, closeness of match may be evaluated between the template and the appropriate heart rate specific enrolled template of the claimed identity.

### Make Decision

The decision making step 608 makes an identification or authentication decision based on all of the results from the previous steps. In identification applications, the decision step may return the determined identity along with an indication of decision confidence In authentication applications, the decision step may return a yes/no decision regarding the validity of an individual's claimed identity. In some embodiments, a decision may not be made or may be deferred due to poor signal quality, not enough information, or poor confidence levels.

Aspects of the invention will now be further demonstrated through reference to the following examples.

### Example #1

### Subject Enrolment Mode

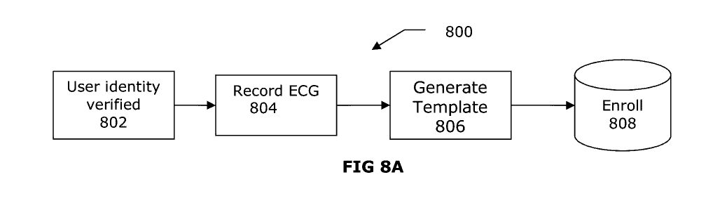

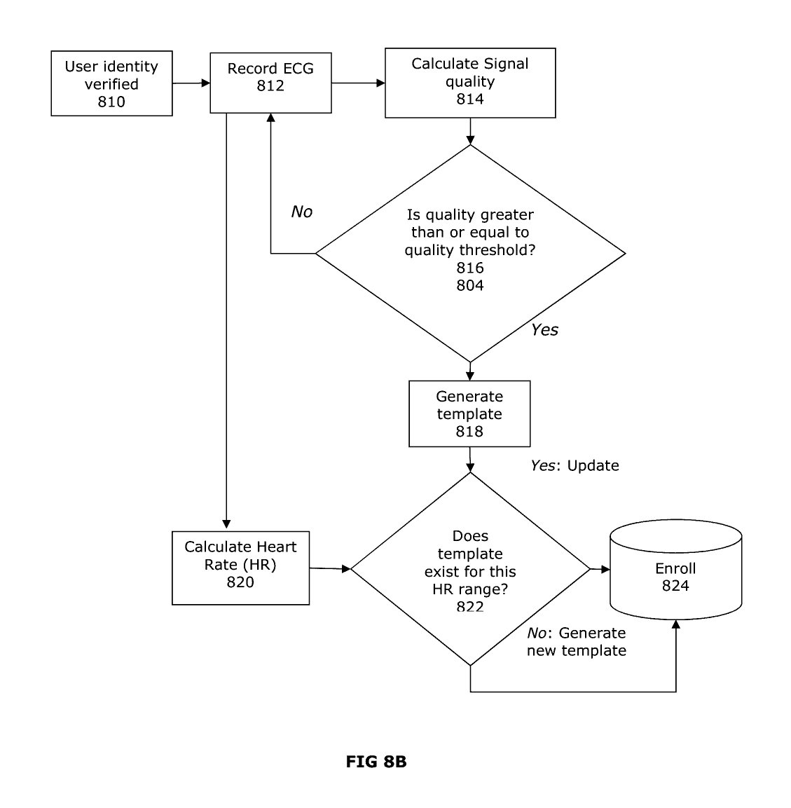

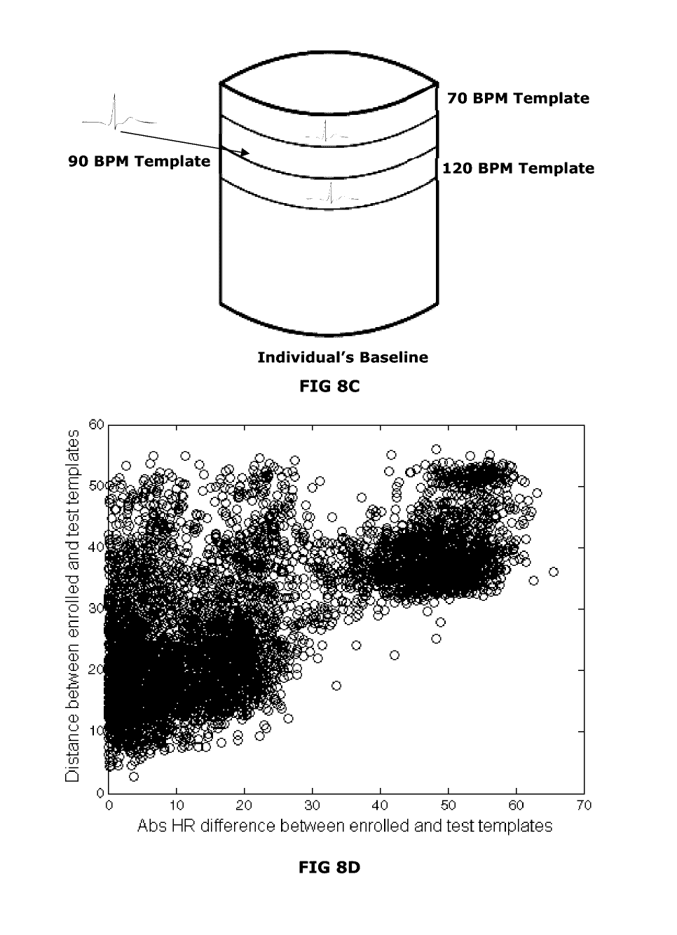

FIG. 8A depicts an embodiment of an ECG biometric method 800 in enrolment mode. Enrolment is the first data collection procedure in a biometric security system, which establishes the benchmark against which an individual will be identified or authenticated.

In this embodiment, ECG templates are generated from a previously-authenticated individual, associated with that individual's identity, and stored in an enrolment database. At enrolment stage, the system recognizes the identity of a person, for example, by official identification documents, and verifies the user's identity 802. The system then establishes base ECG templates of the person at various heart beat rates. The system then associates these base templates with the identity of the person, and registers these base templates with the system for use in the identification or authentication process. These enrolled base templates are later used by the system when an unknown individual requires authentication or identification.

In the basic enrolment procedure, the authenticated subject uses the same ECG recording equipment that he or she will later use for identification or authentication. Several seconds (e.g. 10-30) of ECG signal are then recorded 804, the template generation step 806 is run as described above, and the template is saved 808 in an enrolment database.

FIG. 8B depicts a variation of the basic enrolment method 800, which integrates a signal quality assessment and a heart rate calculation. These additional features may be used in combination, or separately, depending on the needs of the application. In this variation, a user identity is verified 810 as described above and an ECG may be recorded at 812 from the user.

Signal quality may be automatically assessed 814 on the recorded ECG. If the quality of the signal is sufficient 816, the template generation step 818 is run and the template is saved in a database. If the signal quality is insufficient, the ECG recording 812 continues until a sufficient amount of high quality data is recorded. Alternatively, the recording will stop if the high quality data is not available for several attempts. In this case, intervention may be required to check the placement of the ECG sensors. Alternatively, after the ECG recording step 812, the heart rate of the target may be calculated 820 and saved in a template after a decision step 822.

The enrolment procedure 824 may also include generating an array of enrolled templates, to compensate for heart rate related changes in the ECG signal. During the enrolment procedure 824, the subject is instructed to perform exercise of varying intensity to elevate his or her heart rate. If the signal quality is sufficient, a series of base templates are generated and saved at a variety of heart rates during the procedure.

In some implementations involving a heart rate calculation, the system first identifies the heart rate of an individual by determining the number of individual heart beats (PQRST complexes) that occur within a signal segment, and dividing the number by the length of time the segment represents. The heart rate is expressed in beats per minute. The system may only enroll new heart rate specific templates depending on their degree of similarity—for example, an 80 bpm template may be generated only if it is significantly different from a prior 60 bpm template for that individual.

In some cases, the enrolment procedure may also involve automatic re-enrolment of an already authenticated individual. For example, an individual who has been authenticated at one heart rate may be automatically re-enrolled when their heart rate reaches a second level. Authenticated subjects may also be automatically re-enrolled after each successful authentication, at pre-determined time intervals, or in response to the fulfillment of specific conditions as indicated by the sensor systems described above. Templates generated during re-enrolment may be added to the enrolment database for that individual or replace existing templates. For example, as shown in FIG. 8C, a template for an individual's heart rate at 90 BPM may be enrolled in a database for that individual and included in a database that includes multiple other heart rates (for example, 70, 90 and 120 BPM). In some cases, this automatic re-enrolment may further augment the quality of enrolled templates and increase the likelihood that the enrolled templates reflect long term variation of a subject's ECG signal. The automatic re-enrolment procedure may replace lower quality templates if the incoming signal is higher quality, for example a lower SNR template may be replaced with a higher SNR template. Further, if the template and incoming signal are similar in quality, averaging them together to create a new template can reduce some of the noise and thus increase the signal to noise ratio of the template.

### Example #2

### Subject Identification Mode

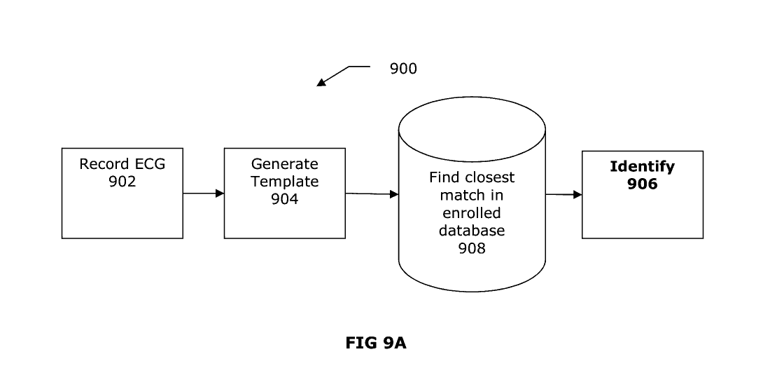

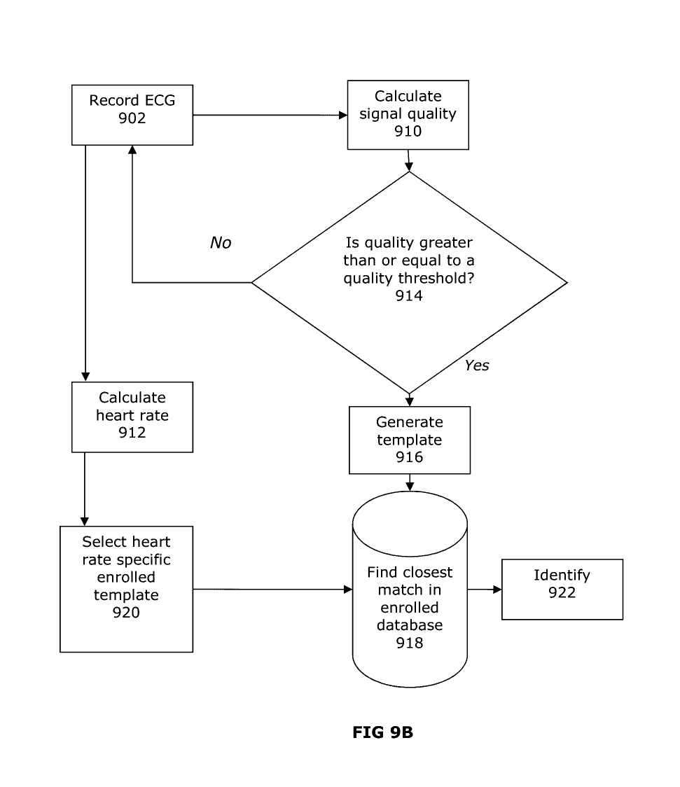

FIG. 9A depicts an embodiment of an ECG biometric method 900 in subject identification mode. In identification mode, the subject is assumed to be enrolled in the database and the system attempts to determine the identity of the subject based on their biometric modality, in this case, an ECG signal. This may be useful in applications where it is necessary to establish ‘proof of life’ for a person of interest (i.e. asset). In basic identification mode, the subject ECG will have already been recorded 902 and the template generation step 904 described will have also been run.

In basic identification mode, the generated ECG template is compared to the base ECG templates of each enrolled individual stored in the database. Comparison measures between the recorded ECG template and the enrolled base template for each enrolled subject are calculated. The similarity between the generated ECG template and each of the stored base ECG templates of each enrolled individual is calculated and a similarity score is generated for each comparison between the generated ECG template and a stored base ECG template of each enrolled individual. Various comparison algorithms may be used in determining the similarity score, including a Wavelet Distance score, cross correlation, Euclidian distance, and Mahalanobis distance. A decision is then made based on the calculated similarity scores to identify 906 the individual by determining the closest match, for example, if the highest similarity score between the generated ECG template and a stored base ECG templates associated with a particular individual in the enrolment database 908 is within a predetermined range, then the particular individual will be identified.

FIG. 9B depicts a variation of the basic identification method, which integrates a signal quality assessment step and a heart rate calculation step 912 after the ECG recording step. These additional features may be used in combination, or separately, depending on the needs of the application.

The ECG signals are collected by sensors and transmitted to a server 506. The server assesses the ECG signal quality automatically on the recorded ECG. If the quality of the signal is sufficient 914, the template generation step 916 is run and the template is used for comparison with the base templates stored in the enrolment database 918. If the signal quality is insufficient, the ECG recording 902 continues until a sufficient amount of high quality data is recorded.

If the enrolled database has an array of templates for each enrolled subject (e.g. representing an array of heart rates), a heart rate calculation may select which of the enrolled templates should be used for comparison 920. The comparisons are made between ECG templates with the same or similar heart rates, which can be subsequently used to identify the target 922. This permits an ECG signal to be compared at the same or similar heart rates, so as to reduce intra-individual variability.

FIG. 8D is a scatter plot illustrating the effect of heart rate on the ability of the system to match a test template against an enrolled template. As illustrated in FIG. 8D, the distribution increases progressively as the difference in BPM increases. This distribution represents the correct matches, the “distance” value for which becomes higher and higher as the heart rate difference increases. There is a wide distribution of scores when the difference in heart rate is less than 10 beats per minute and the system is able to distinguish the differences between the test templates relative to the enrolled templates and find the true matches (who have the low “distance” scores). When the difference in heart rate becomes greater (particularly above 30 beats per minute), the system starts giving the same high distance values to everyone if the difference in beats per minute is too high, essentially rejecting everyone.

### Example #3

### Subject Authentication Mode

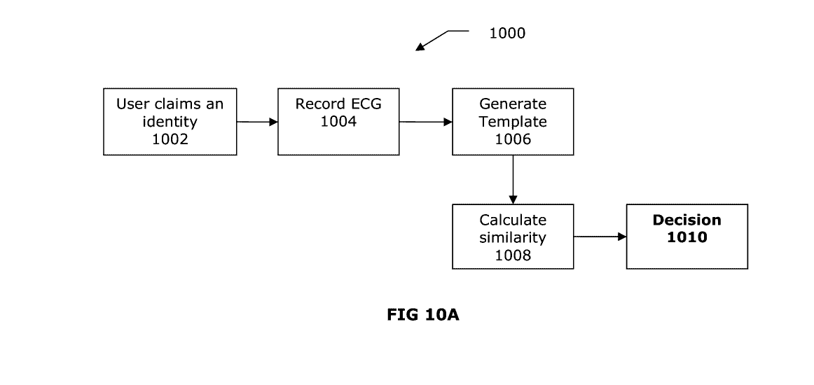

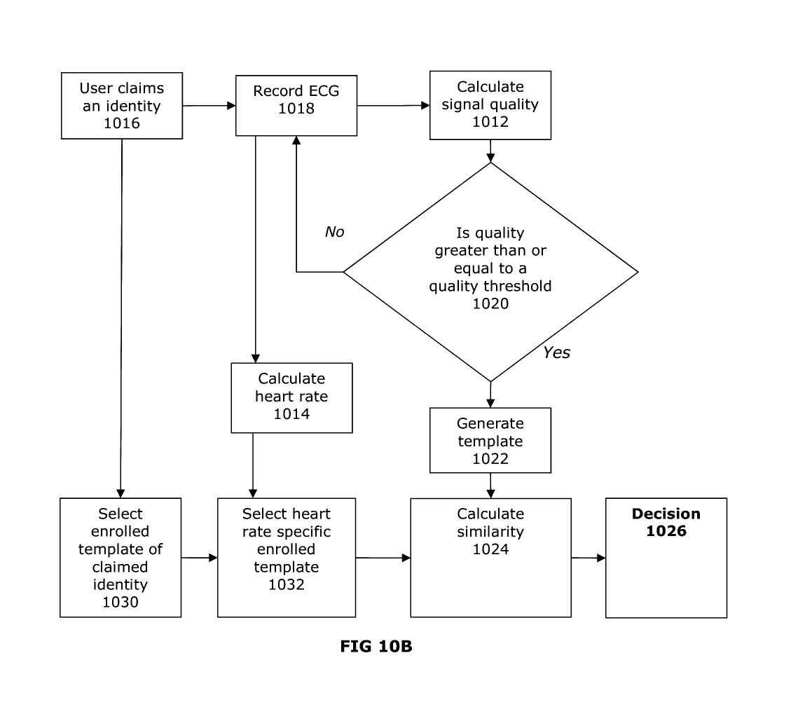

FIG. 10A depicts an embodiment of a biometric identification or authentication method 1000 in subject authentication mode. In authentication mode, a subject claims an identity 1002 using a first authentication factor and the system attempts to confirm or deny this claim using a second modality, such as an ECG signal.

The first authentication factor can take a variety of forms, including entry of a username, password, personal identification number (PIN), or a biometric modality, including a previous authentication using an ECG signal. If a server is used in this mode to authenticate a person, the server stores or has access to the information of the first authentication factor of the person. In an embodiment, the first authentication factor can be obtained at the same time as the ECG signal (for example, a user enters a PIN, and then has to authenticate with an ECG). If the communication device is a cell phone, the first authentication factor can be collected through interaction with the cell phone, such as, by entering a PIN from the cell phone and then transmitting the PIN from the cell phone to the server.

If the first authentication factor has a match in the system, in basic authentication mode, ECG readings are recorded 1004 and the template generation step 1006 is run. The generated ECG template is compared only to the enrolled base templates of the claimed identity based on the first authentication factor, rather than all of the enrolled based templates in the database. The recorded ECG template and the enrolled base templates for the claimed identity with the same or similar heart rate are compared 1008. A decision 1010 is then made to confirm or deny the claimed identity depending on the closeness of the match.

FIG. 10B depicts a variation of the basic authentication method, which integrates a signal quality assessment step 1012 and a heart rate calculation step 1014. These additional features may be used in combination, or separately, depending on the needs of the application. In this embodiment, a user claims an identity 1016 and an ECG is recorded 1018.

Signal quality may be automatically assessed 1012 on the recorded ECG. If the quality of the signal is sufficient as determined at step 1020, the template generation step 1022 is run and the template is used for comparison to the enrolment database 1024 and a decision 1026 is made about the identity of the user. If the signal quality is insufficient, the ECG recording continues until a sufficient amount of high quality data is recorded.

If the enrolled database has an array of templates for each enrolled subject (e.g. representing an array of heart rates), a heart rate calculation step may select 1030 which of the enrolled templates should be used for comparison. This permits ECG signals to be compared 1024 at the same or similar heart rates 1032, so as to reduce intra-individual variability and permit a decision 1026 about the identity of the user.

### Example #4

### Combined Identification/Authentication Mode

In some embodiments, the method operates in a combination of the identification and authentication modes identified above. In such cases, an identification is performed by comparing the measured template of the individual to all of the enrolled base templates in the database and identifying the person by selecting the closest match. Next, an authentication decision is performed to confirm or deny the calculated identity based on the closeness of the match.

This ‘single factor’ authentication method may be less secure than the ‘two-factor’ authentication set out in the authentication mode above. More specifically, in authentication mode, the attacker must first claim an identity (e.g. by circumventing the first authentication factor) before establishing a close match to that claimed identity. In combined mode, the attacker must have an opportunity to emulate a close enough match to any individual in the database.

The methods described herein may be stored in a non-transitory memory and may be performed by a processor.

The embodiments of the present disclosure are intended to be examples only and to accommodate changes in technology. Those of skill in the art may affect alterations, modifications and variations to the particular embodiments without departing from the invention.

In particular, features from one or more of the above-described embodiments may be selected to create alternate embodiments comprised of a sub combination of features which may not be explicitly described above. In addition, features from one or more of the above-described embodiments may be selected and combined to create alternate embodiments comprised of a combination of features which may not be explicitly described above. Features suitable for such combinations and sub combinations would be readily apparent to persons skilled in the art upon review of the present application as a whole. The subject matter described herein and in the recited claims intends to cover and embrace all suitable changes in technology.

### REFERENCES

[1] A. K. Jain, A. Ross, and S. Prabhakar, “An introduction to biometric recognition,” IEEE Trans. Circuits Syst. Video Technol., vol. 14, no. 1, pp. 4-20, January 2004.
[2] Y. Taigman, M. Yang, M. Ranzato, and L. Wolf, “DeepFace: Closing the Gap to Human-Level Performance in Face Verification,” in 2014 IEEE Conference on Computer Vision and Pattern Recognition (CVPR), 2014, pp. 1701-1708.
[3] S. A. Cole, “More than Zero: Accounting for Error in Latent Fingerprint Identification,” J. Crim. Law Criminol. 1973, vol. 95, no. 3, pp. 985-1078, April 2005.
[4] H. W. Draper, C. J. Peffer, F. W. Stallmann, D. Littmann, and H. V. Pipberger, “The Corrected Orthogonal Electrocardiogram and Vectorcardiogram in 510 Normal Men (Frank Lead System),” Circulation, vol. 30, no. 6, pp. 853-864, December 1964.
[5] H. Larkin and S. N. Hunyor, “Precordial voltage variation in the normal electrocardiagram,” J. Electrocardiol., vol. 13, no. 4, pp. 347-351, 1980.
[6] L. S. Green, R. L. Lux, C. W. Haws, R. R. Williams, S. C. Hunt, and M. J. Burgess, “Effects of age, sex, and body habitus on QRS and ST-T potential maps of 1100 normal subjects.,” Circulation, vol. 71, no. 2, pp. 244-253, February 1985.
[7] G. Kozmann, R. L. Lux, and L. S. Green, “Sources of variability in normal body surface potential maps.,” Circulation, vol. 79, no. 5, pp. 1077-1083, May 1989.
[8] A. van Oosterom, R. Hoekema, and G. J. H. Uijen, “Geometrical factors affecting the interindividual variability of the ECG and the VCG,” J. Electrocardiol., vol. 33, Supplement 1, pp. 219-227, 2000.
[9] R. Hoekema, G. J. H. Uijen, and A. van Oosterom, “Geometrical aspects of the interindividual variability of multilead ECG recordings,” IEEE Trans. Biomed. Eng., vol. 48, no. 5, pp. 551-559, May 2001.
[10] T. C. Pilkington, R. C. Barr, and C. L. Rogers, “Effect of conductivity interfaces in electrocardiography,” Bull. Math. Biophys., vol. 30, no. 4, pp. 637-643, December 1968.
[11] B. P. Simon and C. Eswaran, “An ECG Classifier Designed Using Modified Decision Based Neural Networks,” Comput. Biomed. Res., vol. 30, no. 4, pp. 257-272, August 1997.
[12] Z. Zhang and D. Wei, “A New ECG Identification Method Using Bayes' Teorem,” in TENCON 2006. 2006 IEEE Region 10 Conference, 2006, pp. 1-4.
[13] G. Wübbeler, M. Stavridis, D. Kreiseler, R.-D. Bousseljot, and C. Elster, “Verification of humans using the electrocardiogram,” Pattern Recognit. Lett., vol. 28, no. 10, pp. 1172-1175, July 2007.
[14] H. Silva, H. Gamboa, and A. Fred, “One Lead ECG Based Personal Identification with Feature Subspace Ensembles,” in Machine Learning and Data Mining in Pattern Recognition, P. Perner, Ed. Springer Berlin Heidelberg, 2007, pp. 770-783.
[15] A. D. C. Chan, M. M. Hamdy, A. Badre, and V. Badee, “Wavelet Distance Measure for Person Identification Using Electrocardiograms,” IEEE Trans. Instrum. Meas., vol. 57, no. 2, pp. 248-253, February 2008.
[16] I. Odinaka, P.-H. Lai, A. D. Kaplan, J. A. O'Sullivan, E. J. Sirevaag, S. D. Kristjansson, A. K. Sheffield, and J. W. Rohrbaugh, “ECG biometrics: A robust short-time frequency analysis,” in 2010 IEEE International Workshop on Information Forensics and Security (WIFS), 2010, pp. 1-6.
[17] T.-W. (David) Shen, W. J. Tompkins, and Y. H. Hu, “Implementation of a one-lead ECG human identification system on a normal population,” J. Eng. Comput. Innov., vol. 2, no. 1, pp. 12-21, January 2010.
[18] Wahabi, S., S. Pouryayevali, S. Hari, and D. Hatzinakos. “On Evaluating ECG Biometric Systems: Session-Dependence and Body Posture.” IEEE Transactions on Information Forensics and Security 9, no. 11 (November 2014): 2002-13. doi:10.1109/TIFS.2014.2360430.
[19] A. K. Jain, P. Flynn, and A. A. Ross, Handbook of Biometrics, 1st ed. Springer Publishing Company, Incorporated, 2010.
[20] M. Malik, K. Hnatkova, M. Sisakova, and G. Schmidt, “Subject-specific heart rate dependency of electrocardiographic QT, PQ, and QRS intervals,” J. Electrocardiol., vol. 41, no. 6, pp. 491-497, November 2008.
[21] C. E. Garnett, H. Zhu, M. Malik, A. A. Fossa, J. Zhang, F. Badilini, J. Li, B. Darpö, P. Sager, and I. Rodriguez, “Methodologies to characterize the QT/corrected QT interval in the presence of drug-induced heart rate changes or other autonomic effects,” Am. Heart J., vol. 163, no. 6, pp. 912-930, June 2012.
[22] R. L. Donnerstein, D. Zhu, R. Samson, A. M. Bender, and S. J. Goldberg, “Acute effects of caffeine ingestion on signal-averaged electrocardiograms,” Am. Heart J., vol. 136, no. 4, pp. 643-646, October 1998.
[23] J. T. Catalano, Guide to ECG Analysis. Lippincott Williams & Wilkins, 2002.
[24] G. Andrássy, A. Szabo, G. Ferencz, Z. Trummer, E. Simon, and Á. Tahy, “Mental Stress May Induce QT-Interval Prolongation and T-Wave Notching,” Ann. Noninvasive Electrocardiol., vol. 12, no. 3, pp. 251-259, July 2007.
[25] F. Agrafioti, “ECG in Biometric Recognition, Time Dependency and Application Challenges,” Ph.D., University of Toronto, Toronto, ON, 2011.

1. A method of enrolling a living or non-living asset in an asset security system, the method comprising the steps of:
with a processing device, verifying the identity of the asset using at least one prior authentication factor;
acquiring a signal, via one or more sensors, from the asset;
transmitting the signal from the one or more sensors, via a transmission link, to the processing device;
with the processing device, associating the signal with the identity of the asset;
using the processing device to generate an enrolment template for the asset from the signal; and
storing the enrolment template in an enrolment database.

2. The method of claim 1, wherein the asset is a living asset having a heart rate and the signal is an electrocardiogram (ECG) signal at a given heart rate.

3. The method of claim 2, wherein a plurality of templates at a plurality of given heart rates are stored for the asset in the enrolment database

4. The method of claim 3, wherein a plurality of templates at a given heart rate are averaged to generate a new template.

5. A method of authenticating a living or non-living asset, the method comprising the steps of:
with a processing device, verifying the identity of the asset by comparing a first authentication factor with a prior authentication factor;
if a match of the first authentication factor and the prior authentication factor is found, acquiring a signal from the asset, via one or more sensors;
transmitting the signal from the one or more sensors, via a transmission link, to the processing device;
with the processing device, generating a query template for the asset from the signal;
with the processing device, comparing the query template to templates stored in an enrolment database and associated with the asset, verified by the at least one prior authentication factor;
using the processing device to generate a similarity score for each of the templates stored in the enrolment database; and
if a highest similarity score generated is within a predetermined range, authenticating, with the processing device, the asset as an enrolled asset.

6. The method of claim 5, wherein the asset is a living asset having a heart rate and the signal is an electrocardiogram (ECG) signal at a given heart rate.

7. The method of claim 5, wherein the prior authentication factor comprises a personal identification number (PIN), password, physical location, Global Positioning System coordinate, or biometric modality, including an ECG signal.

8. The method of claim 5, wherein the signal of the authenticated asset transmitted to the processing device establishes proof of life of the asset.

9. The method of claim 6, wherein the step of comparing the query template further comprises:
determining the given heart rate for the query template; and
comparing the query template against enrolled templates collected at the given heart rate of the asset.

10. The method of claim 5, wherein the step of acquiring the signal further comprises:
assessing, with the processing device, the quality of the signal to generate a quality measure; and
if the quality measure is less than or equal to a quality threshold, re-acquiring the signal from the asset.

11. A biometric security system comprising:
one or more sensors;
a processing device in communication with the one or more sensors and an enrolment database, the processing device comprising a processor,
wherein the processor is configured to:
verify the identity of an asset by comparing a first authentication factor with a prior authentication factor;
if a match of the first authentication factor and the prior authentication factor is found, receive and acquire a signal from the asset, the signal sent via a transmission link from the one or more sensors;
generate a query template for the asset based on the signal received;
compare the query template to templates stored in the enrolment database and associated with the asset verified by the first authentication factor;
generate a similarity score for each of the templates stored in the enrolment database; and
if a highest similarity score generated is within a predetermined range, authenticate the asset as an enrolled asset.

12. The system of claim 11, wherein the signal is an electrocardiogram (ECG) signal at a given heart rate.

13. The system of claim 12, wherein the ECG signal is acquired from the one or more sensors which are on or associated with a wearable device.

14. The system of claim 11, wherein the processor is further configured to establish proof of life of the asset if the asset is authenticated.

15. The system of claim 11, wherein the processor is further configured to:
determine at least one condition of the asset based on at least one physical or physiological characteristic; and
if the at least one condition of the asset is determined to meet a threshold, issue an instruction to the one or more sensors or to the asset, or issue an alert message.

16. The system of claim 15, wherein the at least one physical or physiological characteristic of the asset is an image, video, physical location, Global Positioning System coordinate, temperature, indicia of assembly status, heart rate, oxygen saturation, or electrocardiogram (ECG) signal.

17. The system of claim 15, wherein the at least one condition of the asset is the identity of the asset, the authentication status of the asset, the location of the asset, or whether the asset has been killed or destroyed.

18. The system of claim 11, wherein the asset is a person and the one or more sensors are associated with the person, the one or more sensors including an ECG sensor, sensing an electrocardiogram signal of the person, and in communication with a transmitter on or associated with the person, the transmitter for transmitting the electrocardiogram signal of the person to the processing device.

19. The system of claim 18, wherein the one or more sensors further include a Global Positioning System sensor, sensing the location of the person, and in communication with the transmitter, permitting the transmitter to transmit the position of the person to the processing device.

20. The system of claim 19, wherein the sensors are in Bluetooth™ communication with the transmitter.
